(19) United States
(12) Patent Application Publication
(10) Pub. No.: US 2009/0247919 A1
(43) Pub. Date: Oct. 1, 2009

(54) Infinitely adjustable loop pile controlled rotation, and locking mechanism and method

(76) Inventor: MARCUS James CASSAR, West Linn, OR (US)

Correspondence Address: MICHAEL RIES, 318 PARKER PLACE, OSWEGO, IL, 60543, US

(21) Appl. No.: 12/412,358

(22) Filed: Mar. 27, 2009

Related U.S. Application Data
(60) Provisional application No. 61/040,644, filed on Mar. 28, 2008.

(51) Int. Cl. A61F 5/00

(52) U.S. Cl. 602/20; 602/36

(57) ABSTRACT

A device for interlocking two sections such as a forearm-hand brace and a elbow-forearm medical device comprising, a circular strip of loop pile or hook fastener material attached to the outer surface of a first cylindrical section such as the custom forearm-hand brace. A sheet of thermo-formable material formed into a second cylindrical section matching the outer surface of the first cylindrical section wherein the second section includes a groove such that the first cylindrical section is held by the circular fastener strip within the groove and is free to rotate in either direction. A cooperating locking strap of hook or loop pile fastener attached to the second section and aligned with the strip of loop pile or hook fastener on the first cylindrical section such that attaching the locking strap to the strip prevents the free rotation and locks the first cylindrical section to the second section.

This application claims the benefit of U.S. Provisional Patent Application Ser. No. 61/040,644, filed Mar. 28, 2008, the entire disclosure of which is incorporated herein by reference.

### BACKGROUND OF THE INVENTION

### Field of the Invention

The invention relates to interlocking cylinders and the means to lock those cylinders in any desired degree of rotation. In some fields it is necessary to adjustably interconnect cylinders. For example, in the field of medicine, it is sometimes necessary to apply an orthopedic brace over the arm of a patient to improve its alignment and function. The orthopedic brace must be adjustable to precisely locate the hand or arm as medically required. Many pathologies cause the muscles in the human forearm to tighten, which can resist or even stop active or passive rotation of the forearm. Since the human hand is attached to the forearm, any limitation of forearm movement therefore limits the positioning of the human hand for functional use. Often, these pathologies cause a patient to maintain their hand and forearm in a pronated (elbow flexed to 90 degrees and palm facing the floor) or supinated (elbow flexed to 90 degrees and palm facing the ceiling) position at rest. Common examples of such pathologies include: brachial plexus injury (BPI), elbow fractures, cerebral palsy (CP), surgical muscle lengthening, and forearm muscle contracture.

Prior art methods of fixing this problem include the application of serial casts or slotted interlocking cylinder braces to a persons arm to rotate and lengthen the tightened muscles until the arm can be rotated through a normal range, either passively or actively.

Serial casts are used to hold the forearm and hand at their end range to permit the forearm muscles to grow and lengthen. By applying a series of these casts a clinician can gradually increase the available range-of-motion (ROM) in the forearm, and improve the functional positioning of the hand. Serial casts have to be reapplied each week by an experienced clinician, which is both costly and time consuming for the patient and medical practice. Furthermore, once the final cast is removed there is nothing to stop the muscles from retightening over time and returning the arm and hand to the original position before the casting was initiated.

Slotted interlocking cylinders and rotational step-lock orthopedic braces also have been used to position and hold the forearm and hand to achieve the same results as repeated serial casts. These devices are limited in that they require screws and mechanical joints to hold the forearm and hand in the desired position and do not allow for small degrees of adjustment, which is vital if you want to obtain full joint range of motion. In addition, the exposed metal components are a hazard to those wearing these orthopedic devices.

As can be seen, there is a need for an improved apparatus and method of interlocking cylinder sections that will allow for a greater range of adjustment, easy re-adjustment, simple application and removal, and minimal mechanical parts.

### BRIEF DESCRIPTION OF THE DRAWINGS

FIG. 1 shows a view of the elbow-forearm medical device 100 with the trough-like partial cylinder 102 and lower arm partial cylinder 104 installed.

FIG. 2 shows a view of the elbow-forearm-hand brace 300 with the forearm-hand brace 200 interlocked with the lower arm partial cylinder 104 and rotated into a pronated position.

FIG. 3 shows a view of the forearm-hand brace 200.

FIG. 4 shows the forearm-hand brace 200 interlocked with the lower arm partial cylinder 104 by the loop pile fastener strap 120 and overlying locking strap 250.

FIG. 5 shows a view of the complete elbow-forearm-hand brace 300.

FIG. 6 shows a view of the complete elbow-forearm-hand brace 300 with the forearm-hand brace 200 interlocked with the lower arm partial cylinder 104 and rotated into a supinated position.

FIG. 7 shows the fabrication flowchart 400, which describes the steps required to make the elbow-forearm-hand brace 300

### DESCRIPTION OF THE PREFERRED EMBODIMENT

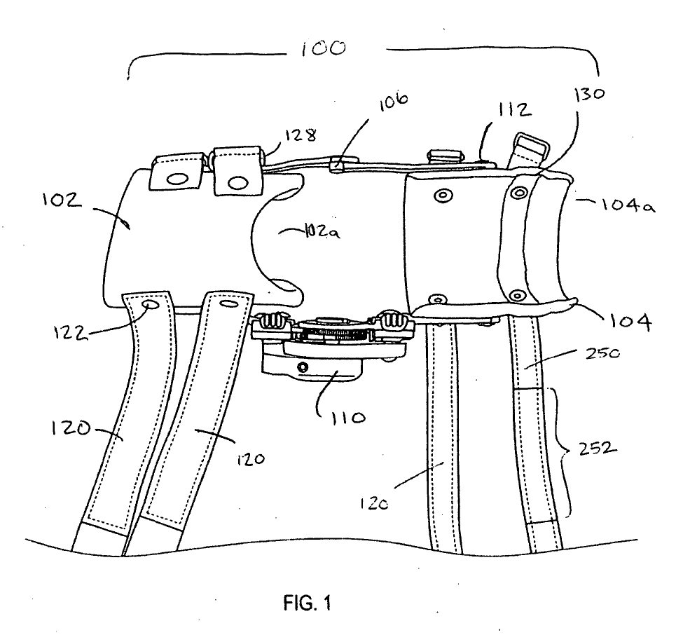

FIG. 1 shows the elbow-forearm section of a medical device that can be applied over the arm of a patient. The elbow-forearm medical device 100 includes a custom-molded trough-like partial cylinder 102 that intimately fits around the patient's upper arm. It will be understood that the use of the term cylinder is used generally to describe a shape that is similar to a cylinder, like a patient's arm. The trough-like partial cylinder 102 is fabricated by lining a cast of the patient's upper arm with a soft, compliant material, such as Plastazote or Aliplast, and then drape molding, under vacuum or another forming process, a piece of thermoplastic material, such as polypropylene, over top of it. The newly formed, lined upper arm cylinder is then cut off the cast and trimmed to form the trough-like partial cylinder. The soft compliant inner material is used to minimize abrasion to the patient's skin during use.

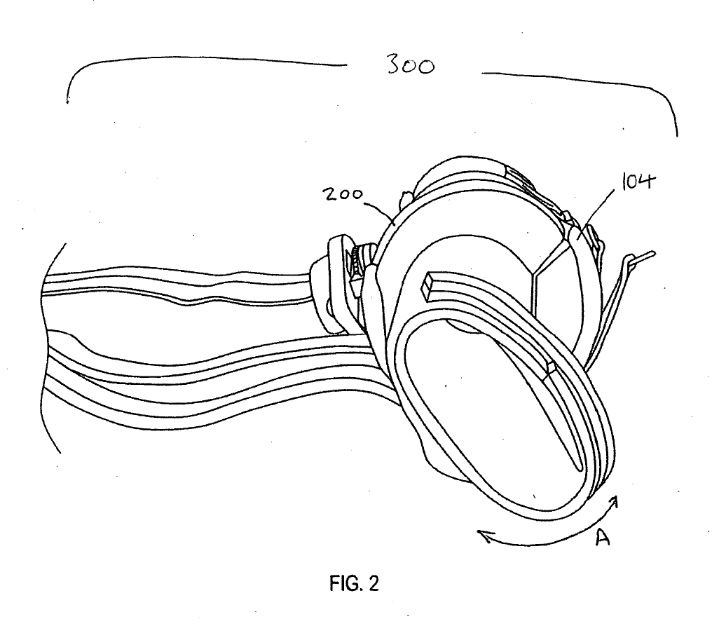

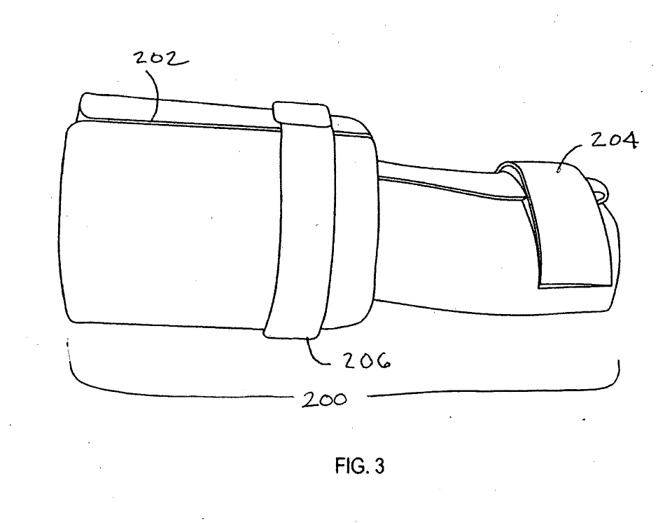

The elbow-forearm medical device 100 also includes a lower arm partial cylinder 104. The elbow-forearm medical device 100 includes a commercial, adjustable orthotic elbow joint 110 that allows the trough-like partial cylinder 102 to be angularly adjusted relative to the lower-arm partial cylinder 104. The lower arm partial cylinder 104 and trough-like partial cylinder 102 are fastened to the elbow-forearm medical device 100 by copper rivets 112. The lower arm partial cylinder 104 is formed by drape molding, under vacuum or another forming process, a piece of thermoplastic material, such as polypropylene, over the previously fabricated forearm-hand brace 200 (FIG. 3) that had been put back on the cast of the patient's forearm and hand. This enables the forearm-hand brace 200 to intimately fit within the lower arm partial cylinder 104 and is able to rotate freely in either direction. The trough-like partial cylinder 102 and lower arm partial cylinder 104 can include multiple loop pile fastener straps 120 attached via rivets 122. The pile-hook fastener straps 120 can include cooperating ‘D’ rings 128 attached to the opposite side of the trough-like partial cylinder 102 and lower arm partial cylinder 104. The trough-like partial cylinder 102 has an opening 102a that allows a patient's upper arm to be placed inside. The lower arm partial cylinder 104 has an opening 104a that allows the forearm-hand brace 200 to be connected to the lower arm partial cylinder 104. The lower arm partial cylinder 104 includes at least one cylindrical groove 130 which, is formed when the lower arm partial cylinder 104 is drape molded, under vacuum or another forming process, over the forearm-hand brace 200. The groove assists the forearm-hand brace 200 to be held within the lower arm partial cylinder 104 and to stop it sliding forward or backward during use. Furthermore, the grove enables the forearm-hand brace 200 to be rotated in either direction about the approximate axis of the lower arm partial cylinder 104. This ability to rotate is shown by the double headed arrow ‘A’ in FIG. 2.

FIG. 3 shows a view of the forearm-hand brace 200. The forearm-hand brace 200 is created in five steps. Step 1, 402 is to line the outside of a cast of the patient's lower arm, wrist and hand with a soft, compliant material, such as Plastazote or Aliplast. The soft compliant inner material is used to minimize friction to the patient's skin during use. Step 2, 404 is to glue and wrap firm thermo-moldable foam, such as Pelite, about the forearm section and grind this outer layer into a cylindrical shape until it has the same diameter from the patient's wrist to the end of the patient's forearm (this ensures that the inside of the forearm-hand brace 200 matches the shape of the patient's arm while the outside is a cylindrical shape to permit rotation). Step 3, 406 is to form the outer plastic skin of the forearm-hand brace 200 by drape molding, under vacuum or another forming process, a thermoplastic material, such as polyethylene, over top of it. Step 4, 408 is to add one or more strips of thermo-moldable foam around the circumference of the forearm-brace 200 to form the corresponding groove(s) 130 in the lower arm partial cylinder 104. Step 5, 410 is to trim off the forearm-hand brace 200 from the underlying cast and form the top opening 202 so that the forearm-hand brace 200 can be applied around the patient's forearm and hand and held closed by loop-pile strap 204.

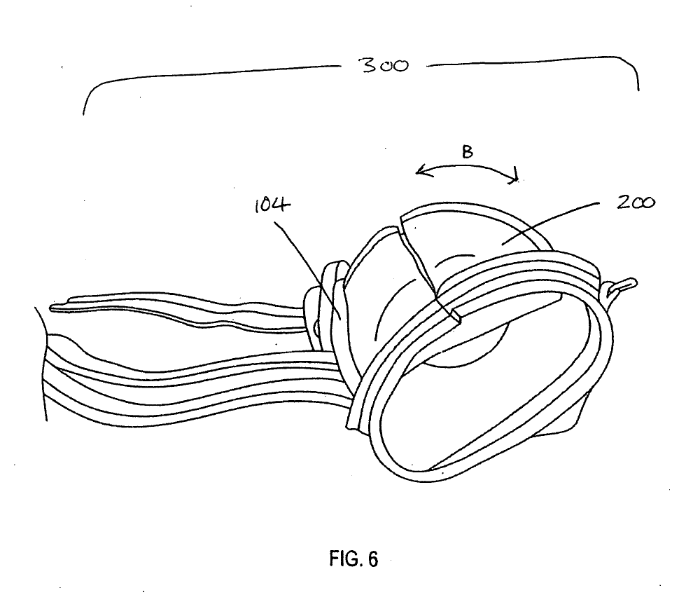

Once the groove(s) 130 have been formed during the fabrication of the lower arm partial cylinder 104 the cylindrical strip(s) of foam are removed from the outside of the forearm-hand brace 200 and are replaced by a adhesive backed strip(s) of pile material 206. The adhesive backed strip(s) of pile material 206 enables the forearm-hand brace 200 to not only, remain within the lower arm partial cylinder 104 but also, be able to rotate either direction with 360 degrees of rotational freedom (FIGS. 2 and 6).

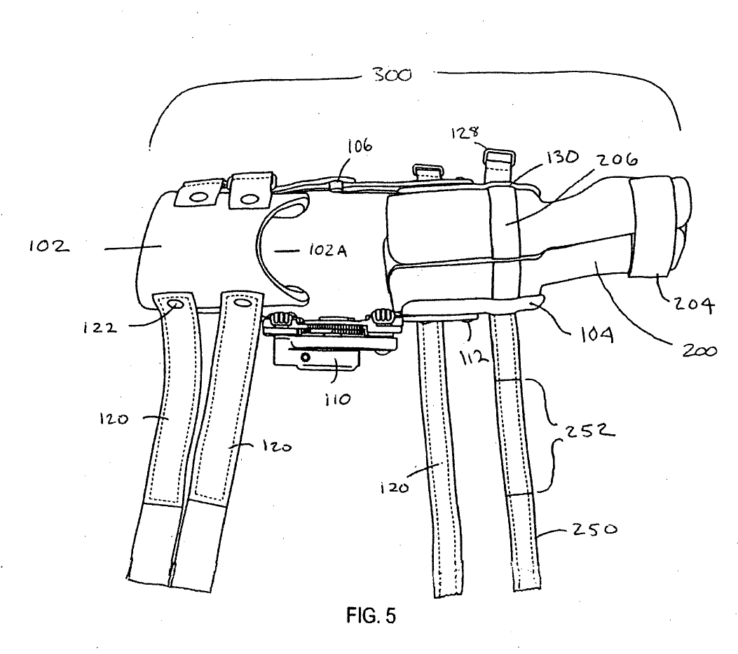

When the overlying locking strap 250 (FIG. 5) with its underlying section of hook material 252 is tightened through the ‘D’ ring 128 it not only, locks the forearm-hand brace 200 into the lower arm partial cylinder 104, but also stops the forearm-hand brace 200 from rotating any direction. If multiple grooves 130 are used on the forearm-hand brace 200 then there will be a corresponding number of adhesive backed strips of pile material 206 and overlying locking straps 250.

The ability to infinitely adjust the rotation position of the forearm-hand brace 200 within the lower arm partial cylinder 104 and then to be able to lock it in place is the essence of the infinitely adjustable loop pile controlled rotation and locking invention.

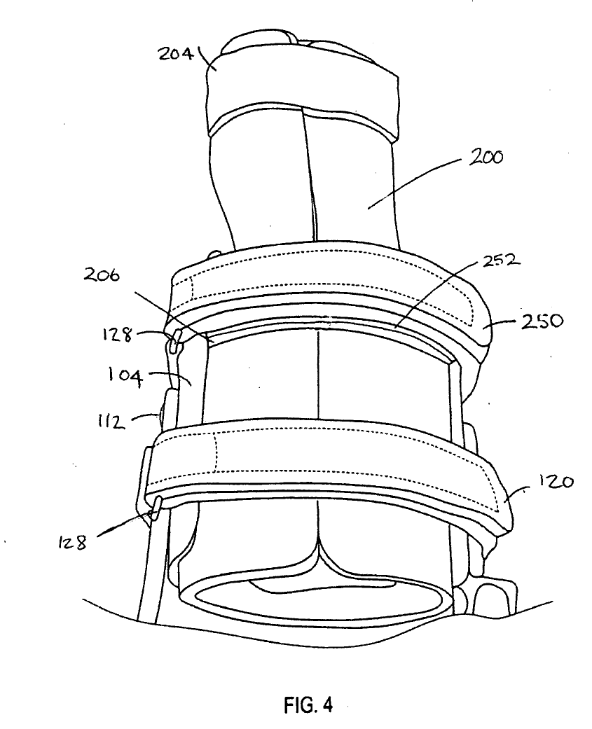

FIG. 4 shows a view of the forearm-hand brace 200 locked down inside the lower arm partial cylinder 104. The remaining parts of the elbow-forearm medical device 100 have been cut away. The loop pile fastener strap 120 has been looped through a ‘D’ ring 128 and fastened back on itself. The overlying locking strap 250 has been looped through a ‘D’ ring 128 and fastened back on itself and its underlying section of hook material 252 has attached to the adhesive backed strip of pile material 206 which runs around the circumference of the forearm-hand brace 200.

FIG. 5 shows a view of the elbow-forearm-hand brace 300 which is made by placing the forearm-hand brace 200 inside of the elbow-forearm medical device 100. The overlying locking strap 250 includes an underlying section of hook material 252 that will adhere to the corresponding adhesive backed strip of pile material 206 that has been fastened around the forearm-hand brace 200.

FIG. 6 shows a view of the complete elbow-forearm-hand brace 300 with the forearm-hand brace 200 interlocked with the lower arm partial cylinder 104 and rotated into a supinated position. The double arrow ‘B’ indicates that the forearm-hand brace 200 can be rotated in either direction from its current position.

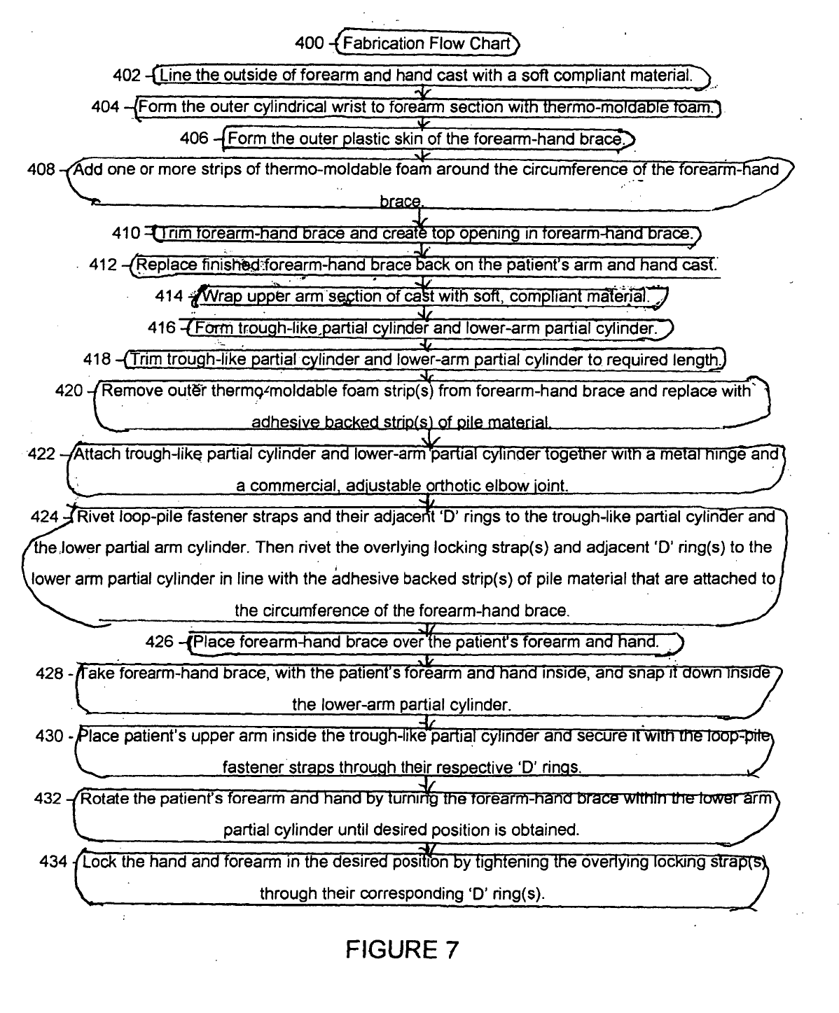

FIG. 7 shows the fabrication flowchart 400, which describes the steps required to make the elbow-forearm-hand brace 300. The elbow-forearm-hand brace 300 is made up of two interconnecting parts: the elbow-forearm medical device 100 and the forearm-hand brace 200. There are 17 fabrication steps in all. Step 1, 402 is to line the outside of a cast of the patient's lower arm, wrist and hand with a soft, compliant material, such as Plastazote or Aliplast. The soft compliant inner material is used to minimize friction to the patient's skin during use. Step 2, 404 is to glue and wrap firm thermo-moldable foam, such as Pelite, about the forearm section and grind this outer layer into a cylindrical shape until it has the same diameter from the patient's wrist to the end of the patient's forearm (this ensures that the inside of the forearm-hand brace 200 matches the shape of the patient's arm while the outside is a cylindrical shape to permit rotation). Step 3, 406 is to form the outer plastic skin of the forearm-hand brace 200 by drape molding, under vacuum or other forming process, a thermoplastic material, such as polyethylene, over top of it. Step 4, 408 is to add one or more strips of thermo-moldable foam around the circumference of the forearm-brace 200 to form the corresponding groove(s) 130 in the lower arm partial cylinder 104. Step 5, 410 is to trim off the forearm-hand brace 200 from the underlying cast and form the top opening 202 so that the forearm-hand brace 200 can be applied around the patient's forearm and hand and held closed by loop-pile strap 204. Step 6, 412 is to replace the completed forearm-hand brace 200 back over the cast of the patient's arm and hand. Step 7, 414 is to wrap the upper arm section of the patient's cast with a soft, compliant material, such as Plastazote or Aliplast, and trim it to length. Step 8, 416 is to form the trough-like partial cylinder 102 and the lower arm partial cylinder 104 by drape molding, under vacuum or by another forming process a piece of thermoplastic material, such as polypropylene, over the top of the soft material upper arm section and the forearm-hand brace 200. Step 9, 418 is to remove the newly formed trough-like partial cylinder 102 and the lower arm partial cylinder 104 from the cast and the forearm-hand brace 200 and trim them to size. Step 10, 420 is to remove the strips of thermo-moldable foam from the outside of the forearm-hand brace 200 and replace them with adhesive backed strip(s) of pile material 206. Step 11, 422 is to attach a metal hinge 106 (FIG. 1) and a commercial, adjustable orthotic elbow joint 110 to the trough-like partial cylinder 102 and the lower arm partial cylinder 104 with rivets 112. By connecting the trough-like partial cylinder 102 to the lower arm partial cylinder 104 it forms the elbow-forearm medical device 100. Step 12, 424 is to rivet 112 the loop-pile fastener straps 120 and their adjacent ‘D’ rings 128 to the trough-like partial cylinder 102 and the lower partial arm cylinder 104, and to rivet 112 the overlying locking strap(s) 250 and adjacent ‘D’ ring(s) to the lower arm partial cylinder 104 in line with the adhesive backed strip(s) of pile material 206 that are attached to the circumference of the forearm-hand brace 200. Step 13, 426 is to attach the forearm-hand brace 200 over the patient's forearm and hand. Step 14, 428 is to take the forearm-hand brace 200, with the patient's forearm and hand inside, and snap it down inside the lower-arm partial cylinder 104. Step 15, 430 is to take the patient's upper arm and place it inside of the trough-like partial cylinder 102 and secure it with the loop-pile fastener straps 120 through their respective ‘D’ rings 128. Step 16, 432 is to rotate the patient's forearm and hand by turning the forearm-hand brace 200 within the lower arm partial cylinder 104 until the desired position is obtained. Step 17, 434 is to lock the hand and forearm in the desired position by feeding the overlying locking strap(s) 250 through their corresponding ‘D’ rings 128 and tightening them down so that the underlying section of hook material 252 adheres to the corresponding adhesive backed strip of pile material 206 that is around the forearm-hand brace 200.

It will be obvious to those skilled in the art that modifications may be made to the embodiments described above without departing from the scope of the invention. Thus the scope of the invention should be determined by the claims in the formal application and their legal equivalents, rather than by the examples given.

1. A elbow-forearm-hand brace with interlocking two cylinder sections that are a elbow-forearm medical device and a forearm-hand brace comprising: an adhesive backed strip of pile material that is circumferentially attached to an outer surface of a first cylindrical section; a sheet of thermo-moldable material that is formed over the first cylindrical section so that it creates a matching outer second cylindrical section, wherein an outer groove is formed that matches a placement of the adhesive backed strip of pile material on the first (inner) cylindrical section such that the first (inner) cylindrical section is free to rotate in either direction within the second (outer) cylindrical section, a cooperating overlying locking strap with an underlying section of hook material that is attached to the second (outer) cylindrical section and aligned with the adhesive backed strip of pile material that is circumferentially attached to the outer surface of the first cylindrical section such that attaching the overlying locking strap to the adhesive backed strip of pile material on the first inner cylinder prevents the movement of the first (inner) cylindrical section within the second (outer) cylindrical section.

2. The device of claim 1 wherein the first cylindrical section is a forearm-hand brace applied over a patient's forearm and hand and the second cylindrical section is a lower arm partial cylinder that is part of an elbow-forearm medical device such that a patient wearing the forearm-hand brace can have their forearm and hand locked inside of the lower arm partial cylinder and be rotated in either direction until the overlying locking strap is attached to the adhesive backed strip of pile material that is circumferentially attached to an outer surface of the forearm-hand brace.

3. The device of claim 2 wherein the forearm-hand brace includes a third cylindrical section that is a trough-like partial cylinder, with attached loop pile fastener straps and corresponding ‘D’ rings to hold the elbow-forearm medical device on the upper arm of the patient.

4. The device of claim 2 wherein the second cylindrical section, the lower arm partial cylinder, includes an opening such that it can flex open to permit the first cylindrical section, the forearm-hand brace, to be placed inside and then flex closed afterwards securing the adhesive backed strip of pile material attached to a circumference of the forearm-hand brace within a cooperating cylindrical groove that has been molded into the second cylindrical section and the lower arm partial cylinder.

5. The device of claim 2 wherein the second (outer) cylindrical section, the lower arm partial cylinder, includes a ‘D’ ring adjacent a formed cylindrical groove such that the overlying locking strap is first secured to the adhesive backed strip of pile material attached to a circumference of the forearm-hand brace and then looped through the ‘D’ ring and secured back on itself.

6. The device of claim 2 wherein the forearm-hand brace includes an angular adjustment between the second cylindrical section, the lower-arm partial cylinder and a third cylindrical section that is a trough-like partial cylinder.

7. The method of fabricating an elbow-forearm-hand brace comprising:
lining an outside of a cast of the patient's lower arm, wrist and hand with a soft, compliant material, such as Plastazote or Aliplast, the soft compliant inner material is used to minimize friction to the patient's skin during use;
gluing and wrapping firm thermo-moldable foam, such as Pelite, about a forearm section and grinding this outer layer into a cylindrical shape until it has a same diameter from the patient's wrist to the end of the patient's forearm this ensures that an inside of a forearm-hand brace matches the shape of the patient's arm while an outside is a cylindrical shape to permit rotation;
forming an outer plastic skin of the forearm-hand brace by drape molding, under vacuum or another forming process, a thermoplastic material, such as polyethylene, over a top of the forearm-hand brace;
adding one or more strips of thermo-moldable foam around the circumference of the forearm-brace to form at least one corresponding groove in a lower arm partial cylinder;
trimming off the forearm-hand brace from the underlying cast and form a top opening so that the forearm-hand brace can be applied around the patient's forearm and hand and held closed by loop-pile strap;
replacing the completed forearm-hand brace back over the cast of the patient's arm and hand;
wrapping the upper arm section of the patient's cast with a soft, compliant material, such as Plastazote or Aliplast, and trimming it to length;
forming a trough-like partial cylinder and a lower arm partial cylinder by drape molding, under vacuum or by another forming process a piece of thermoplastic material, such as polypropylene, over the top of a soft material upper arm section and the forearm-hand brace;
removing the newly formed trough-like partial cylinder and the lower arm partial cylinder from the cast and the forearm-hand brace and trimming them to size;
removing strips of thermo-moldable foam from the outside of the forearm-hand brace and replacing them with at least one adhesive backed strip of pile material;
attaching a metal hinge and a commercial, adjustable orthotic elbow joint to the trough-like partial cylinder and the lower arm partial cylinder with rivets, by connecting the trough-like partial cylinder to the lower arm partial cylinder it forms an elbow-forearm medical device;
riveting at least one loop-pile fastener strap and their adjacent ‘D’ rings to the trough-like partial cylinder and the lower partial arm cylinder, and riveting at least one overlying locking strap and adjacent ‘D’ ring to the lower arm partial cylinder in line with the adhesive backed strip of a pile material that are attached to the circumference of the forearm-hand brace.
attaching the forearm-hand brace over the patient's forearm and hand;
taking the forearm-hand brace, with the patient's forearm and hand inside, and snap it down inside the lower-arm partial cylinder;
taking the patient's upper arm and placing it inside of the trough-like partial cylinder and securing it with the loop-pile fastener straps through their respective ‘D’ rings;
rotating the patient's forearm and hand by turning the forearm-hand brace within the lower arm partial cylinder until the desired position is obtained; and
locking the hand and forearm in the desired position by feeding the at least one overlying locking strap through their corresponding ‘D’ rings and tightening them down so that an underlying section of hook material adheres to the corresponding adhesive backed strip of pile material that is around the forearm-hand brace.

8. The method of claim 7 wherein the steps of forming the forearm-hand brace includes the step of trimming the forearm-hand brace to a required length.

9. The method of claim 7 wherein the step of attaching the adhesive backed strip of pile material circumferentially around the forearm-hand brace includes the step of attaching a ‘D’ ring adjacent to a cylindrical groove on an outside of the lower arm partial cylinder.

10. The method of claim 7 wherein the steps of placing the forearm-hand brace within the lower arm partial cylinder includes securing the trough-like partial cylinder to the upper arm of the patient.

11. The method of claim 7 wherein the step of attaching the lower arm partial cylinder to the trough-like partial cylinder involves the attachment of a metal hinge and a commercial, adjustable orthotic elbow joint with rivets.

12. The method of claim 11 including the step of adjusting an angle between trough-like partial cylinder and the lower arm partial cylinder with the use of the commercial, adjustable orthotic elbow joint.
